(19) United States
(12) Patent Application Publication
(10) Pub. No.: US 2025/0049564 A1
(43) Pub. Date: Feb. 13, 2025

(54) DEVICES, SYSTEMS AND METHODS FOR IMPROVING RECAPTURE OF PROSTHTIC HEART VALVE DEVICE WITH STENT FRAME HAVING VALVE SUPPORT WITH INWARDLY STENT CELLS

(71) Applicant: 4C Medical Technologies, Inc., Maple Grove, MN (US)

(72) Inventors: Jonathan Berndt, Crystal, MN (US); Sounthara Khouengboua, Chaska, MN (US); Saravana B. Kumar, Minnetonka, MN (US); Jaqueline Wendel, Hopkins, MN (US); Jason S. Diedering, Minneapolis, MN (US)

(21) Appl. No.: 18/639,053

(22) Filed: Apr. 18, 2024

Related U.S. Application Data
(63) Continuation of application No. 17/111,736, filed on Dec. 4, 2020, now Pat. No. 11,992,403.
(60) Provisional application No. 62/986,151, filed on Mar. 6, 2020.
(60) Provisional application No. 62/987,413, filed on Mar. 10, 2020.
(60) Provisional application No. 62/988,421, filed on Mar. 12, 2020.
(60) Provisional application No. 62/989,209, filed on Mar. 13, 2020.

(51) Int. Cl. A61F 2/24; A61F 2/95

(52) U.S. Cl. CPC A61F 2/2418; A61F 2/2427; A61F 2/95; A61F 2/2412; A61F 2/2439; A61F 2002/9511

(57) ABSTRACT

A collapsible and expandable prosthetic mitral valve stent is provided with improved recapture into a distal lumen of a delivery sheath. Various embodiments comprise a valve support within the interior of a stent frame and defining a flow channel therethrough, wherein the top or upstream of the valve support comprises a row, or a plurality, of stent cells that are bent radially inward at least partially over the flow channel. Some embodiments may comprise a recapture assist mechanism, such as an open paddle, attached to one or more of the inwardly bent stent cells and adapted to receive and/or engage a wire to aid in positioning, expansion, recapture and/or implanting the device in a patient's heart chamber.

### CROSS-REFERENCE TO RELATED APPLICATIONS

This application claims the benefit of U.S. Provisional Application No. 62/986,151, filed Mar. 6, 2020 and entitled DEVICES, SYSTEMS AND METHODS FOR IMPROVING RECAPTURE OF PROSTHETIC HEART VALVE DEVICE WITH STENT FRAME HAVING VALVE SUPPORT WITH INWARDLY STENT CELLS, U.S. Provisional Application No. 62/987,413, filed Mar. 10, 2020 and entitled DEVICES, SYSTEMS AND METHODS FOR IMPROVING RECAPTURE OF PROSTHETIC HEART VALVE DEVICE WITH STENT FRAME HAVING VALVE SUPPORT WITH ELONGATED SECTION, U.S. Provisional Application No. 62/988,421, filed Mar. 12, 2020 and entitled DEVICES, SYSTEMS AND METHODS FOR IMPROVING RECAPTURE OF PROSTHETIC HEART VALVE DEVICE WITH STENT FRAME HAVING VALVE SUPPORT WITH INWARDLY BENT STRUTS and U.S. Provisional Application No. 62/989,209, filed Mar. 13, 2020 and entitled DEVICES, SYSTEMS AND METHODS FOR IMPROVING RECAPTURE OF PROSTHETIC HEART VALVE DEVICE WITH STENT FRAME HAVING VALVE SUPPORT WITH ATTACHMENT TO THE OUTER STENT, the entirety of which are hereby incorporated by reference.

### STATEMENT REGARDING FEDERALLY SPONSORED RESEARCH OR DEVELOPMENT

Not Applicable

### BACKGROUND OF THE INVENTION

### Field of the Invention

The invention relates to devices, systems and features for improving collapsing and/or recapturing efficiency for a collapsible and expandable prosthetic heart valve device.

### Description of the Related Art

The human heart comprises four chambers and four heart valves that assist in the forward (antegrade) flow of blood through the heart. The chambers include the left atrium, left ventricle, right atrium and right ventricle. The four heart valves include the mitral valve, the tricuspid valve, the aortic valve and the pulmonary valve. See generally FIG. 1.

The mitral valve is located between the left atrium and left ventricle and helps control the flow of blood from the left atrium to the left ventricle by acting as a one-way valve to prevent backflow into the left atrium. Similarly, the tricuspid valve is located between the right atrium and the right ventricle, while the aortic valve and the pulmonary valve are semilunar valves located in arteries flowing blood away from the heart. The valves are all one-way valves, with leaflets that open to allow forward (antegrade) blood flow. The normally functioning valve leaflets close under the pressure exerted by reverse blood to prevent backflow (retrograde) of the blood into the chamber it just flowed out of. For example, the mitral valve when working properly provides a one-way valving between the left atrium and the left ventricle, opening to allow antegrade flow from the left atrium to the left ventricle and closing to prevent retrograde flow from the left ventricle into the left atrium. This retrograde flow, when present, is known as mitral regurgitation or mitral valve regurgitation. As is shown, normal blood flow proceeds through the mitral valve from the left atrium to the left ventricle impinging on the posterior lateral side of the left ventricle (as opposed to the septal side). This natural flow takes advantage of the left ventricular anatomy so that the flow is further directed downward within the left ventricle and then upward toward the aortic valve and the left ventricular outflow tract (LVOT) and the associated aortic root, and ultimately into the ascending aorta as shown. Generally, this flow in normal patients is laminar and, therefore, an efficient mechanism.

FIG. 2 illustrates the relationship between the left atrium, annulus, chordae tendineae and the left ventricle relative to the mitral valve leaflets. As is shown, the upper surface of the annulus forms at least a portion of the floor or lower surface of the left atrial chamber, so that for purposes of description herein, the upper surface of the annulus is defined as marking the lower boundary of the left atrial chamber.

Native heart valves may be, or become, dysfunctional for a variety of reasons and/or conditions including but not limited to disease, trauma, congenital malformations, and aging. These types of conditions may cause the valve structure to fail to close properly resulting in regurgitant retrograde flow of blood from the left ventricle to the left atrium in the case of a mitral valve failure. FIG. 3 illustrates regurgitant blood flow with an exemplary dysfunctional mitral valve.

Mitral valve regurgitation is a specific problem resulting from a dysfunctional mitral valve that allows at least some retrograde blood flow back into the left atrium from the right atrium. In some cases, the dysfunction results from mitral valve leaflet(s) that prolapse up into the left atrial chamber, i.e., above the upper surface of the annulus instead of connecting or coapting to block retrograde flow. This backflow of blood places a burden on the left ventricle with a volume load that may lead to a series of left ventricular compensatory adaptations and adjustments, including remodeling of the ventricular chamber size and shape, that vary considerably during the prolonged clinical course of mitral regurgitation.

Regurgitation can be a problem with native heart valves generally, including tricuspid, aortic and pulmonary valves as well as mitral valves.

Native heart valves generally, e.g., mitral valves, therefore, may require functional repair and/or assistance, including a partial or complete replacement. Such intervention may take several forms including open heart surgery and open heart implantation of a replacement heart valve. See e.g., U.S. Pat. No. 4,106,129 (Carpentier), for a procedure that is highly invasive, fraught with patient risks, and requiring not only an extended hospitalization but also a highly painful recovery period.

Less invasive methods and devices for replacing a dysfunctional heart valve are also known and involve percutaneous access and catheter-facilitated delivery of the replacement valve. Most of these solutions involve a replacement heart valve attached to a structural support such as a stent, commonly known in the art, or other form of wire network designed to expand upon release from a delivery catheter. Sec, e.g., U.S. Pat. No. 3,657,744 (Ersek); U.S. Pat. No. 5,411,552 (Andersen). The self-expansion variants of the supporting stent assist in positioning the valve, and holding the expanded device in position, within the subject heart chamber or vessel. This self-expanded form also presents problems when, as is often the case, the device is not properly positioned in the first positioning attempt and, therefore, must be recaptured and positionally adjusted. This recapturing process in the case of a fully, or even partially, expanded device requires re-collapsing the device to a point that allows the operator to retract the collapsed device back into a delivery sheath or catheter, adjust the inbound position for the device and then re-expand to the proper position by redeploying the positionally-adjusted device distally out of the delivery sheath or catheter. Collapsing the already expanded device is difficult because the expanded stent or wire network is generally designed to achieve the expanded state which also resists contractive or collapsing forces.

Besides the open heart surgical approach discussed above, gaining access to the valve of interest is achieved percutaneously via one of at least the following known access routes: transapical; transfemoral; transatrial; and transseptal delivery techniques.

Generally, the art is focused on systems and methods that, using one of the above-described known access routes, allow a partial delivery of the collapsed valve device, wherein one end of the device is released from a delivery sheath or catheter and expanded for an initial positioning followed by full release and expansion when proper positioning is achieved. Sec, e.g., U.S. Pat. No. 8,852,271 (Murray, III); U.S. Pat. No. 8,747,459 (Nguyen); U.S. Pat. No. 8,814,931 (Wang); U.S. Pat. No. 9,402,720 (Richter); U.S. Pat. No. 8,986,372 (Murray, III); and U.S. Pat. No. 9,277,991 (Salahich); and U.S. Pat. Pub. Nos. 2015/0272731 (Racchini); and 2016/0235531 (Ciobanu).

In addition, all known prosthetic heart valves are intended for full replacement of the native heart valve. Therefore, these replacement heart valves, and/or anchoring or tethering structures, physically extend out of the left atrial chamber, in the case of mitral valves, and engage the inner annulus and/or valve leaflets, in many cases pinning the native leaflets against the walls of the inner annulus, thereby permanently eliminating all remaining functionality of the native valve and making the patient completely reliant on the replacement valve. In other cases, the anchoring structures extend into the left ventricle and may anchor into the left ventricle wall tissue and/or the sub-annular surface at the top of the left ventricle. Others may comprise a presence in, or engagement with, a pulmonary artery.

Obviously, there will be cases when native valve has lost virtually complete functionality before the interventional implantation procedure. In this case the preferred solution will comprise an implant that does not extent outside of, e.g., the left atrium, and that functions to completely replace the native valve function. However, in many other cases, the native valve remains functional to an extent and may, or may not, continue to lose functionality after the implantation procedure. A preferred solution in this case comprises delivery and implantation of a valve device that will function both as a supplemental or augmentation valve without damaging the native leaflets in order to retain native valve leaflet functionality as long as present, while also being fully capable of replacing the native function of a valve that slowly loses most or all of its functionality post-implantation of the prosthetic valve.

Finally, known prosthetic cardiac valves consist of two or three leaflets that are arranged to act as a one-way valve, permitting fluid flow therethrough in the antegrade direction while preventing retrograde flow. The native mitral valve is located retrosternally at the fourth costal cartilage, consisting of an anterior and posterior leaflet, chordae tendinae, papillary muscles, ventricular wall and annulus connected to the atria. Each native leaflet is supported by chordac tendinae that are attached to papillary muscles which become taut with each ventricular contraction preserving valvular competence. Both the anterior and posterior leaflets of the native valve are attached via primary, secondary and tertiary chordae to both the antero-lateral and posterio-medial papillary muscles. A disruption in either papillary muscle in the setting of myocardial injury, can result in dysfunction of either the anterior or posterior leaflet of the mitral valve. Other mechanisms may result in failure of one, or both of the native mitral leaflets. In the case of a single mitral valve leaflet failure, the regurgitation may take the form of a non-central, eccentric jet of blood back into the left atrium. Other leaflet failures may comprise a more centralized regurgitation jet. Known prosthetic valve replacements generally comprise leaflets which are arranged to mimic the native valve structure, which may over time become susceptible to similar regurgitation outcomes.

Known implantable prosthetic valves may be improved upon by employing structures that may aid in the recapture of the devices into the lumen of a delivery sheath.

Certain inventive embodiments described herein are readily applicable to single or two chamber solutions, unless otherwise indicated. Moreover, certain embodiments discussed herein may be applied to preservation and/or replacement of native valve functionality generally, with improved native leaflet coaptation and/or prolapsing, and are not, therefore, limited to the mitral valve and may be extended to include devices and methods for treating the tricuspid valve, the aortic valve and/or pulmonary valves.

Various embodiments of the several inventions disclosed herein address these, inter alia, issues.

### BRIEF DESCRIPTION OF THE SEVERAL VIEWS OF THE DRAWINGS

FIG. 1 illustrates certain features of the heart in cross-section.

FIG. 2 illustrates a cross-sectional perspective view of the left side of the heart.

FIG. 3 illustrates a cross-sectional view of the heart showing retrograde blood flow resulting from mitral valve regurgitation compared with normal blood flow.

FIG. 4 illustrates a perspective view of a prosthetic heart valve device.

FIG. 5 illustrates side and partial cutaway view of a prosthetic heart valve device.

FIG. 6 illustrates a perspective view of one embodiment of a prosthetic heart valve device of the present invention.

FIG. 7 illustrates a cutaway view of one embodiment of a prosthetic heart valve device of the present invention.

FIG. 8 illustrates a perspective view of one embodiment of a prosthetic heart valve device of the present invention.

FIG. 9A illustrates a cutaway view of one embodiment of a prosthetic heart valve device of the present invention.

FIG. 9B illustrates one embodiment of a prosthetic heart valve device of the present invention.

FIG. 10 illustrates a cutaway view of one embodiment of a prosthetic heart valve device of the present invention.

FIG. 11 illustrates a cutaway view of one embodiment of a prosthetic heart valve device of the present invention.

FIG. 12 illustrates a side and partial cutaway view of a prosthetic heart valve device of the present invention.

FIG. 13 illustrates a cutaway view of one embodiment of a prosthetic heart valve device of the present invention.

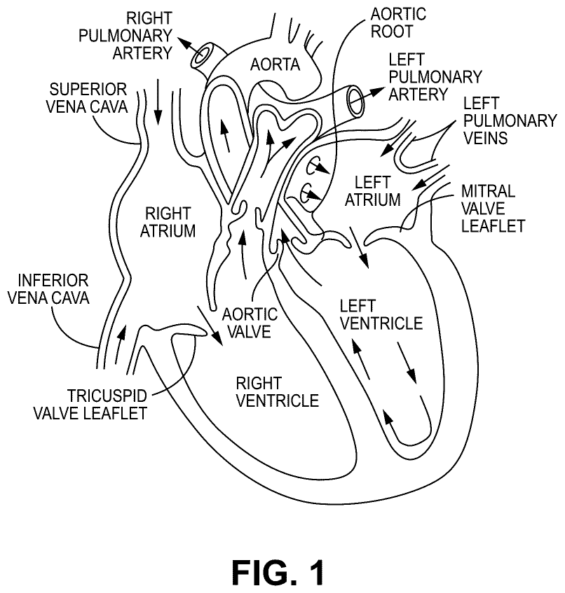

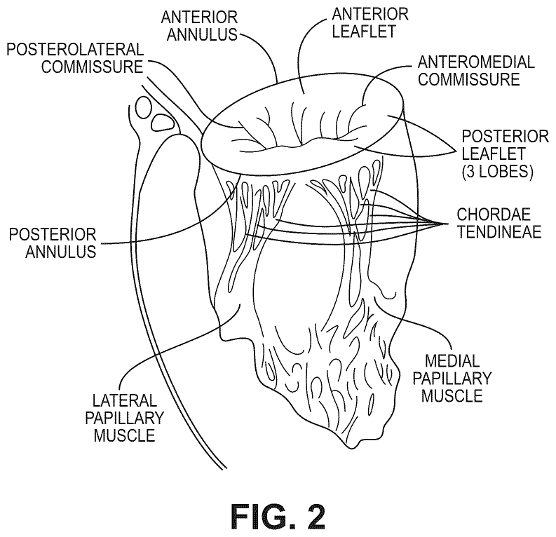

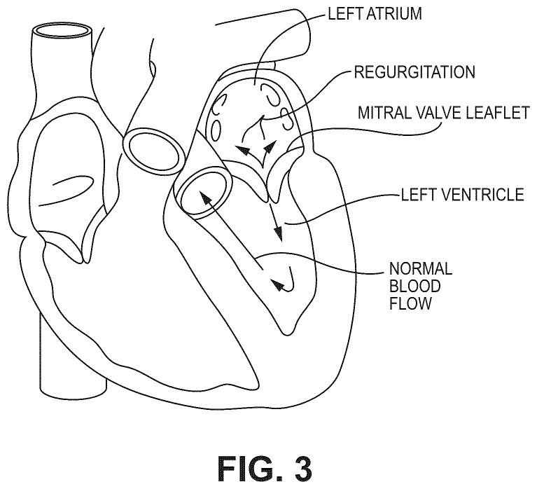

### DETAILED DESCRIPTION OF THE INVENTION

Generally, various embodiments of the present invention are directed to devices and methods for recapture into a distal lumen of a delivery sheath during the expansion and implantation processes. Various embodiments comprise a valve support within the interior of a stent frame and defining a flow channel therethrough, wherein the top or upstream of the valve support comprises a row of stent cells that are bent radially inward at least partially over the flow channel. Some embodiments may comprise a recapture assist mechanism, such as an open paddle, attached to one or more of the inwardly bent stent cells and adapted to receive and/or engage a wire to aid in positioning, expansion, recapture and/or implanting the device in a patient's heart chamber.

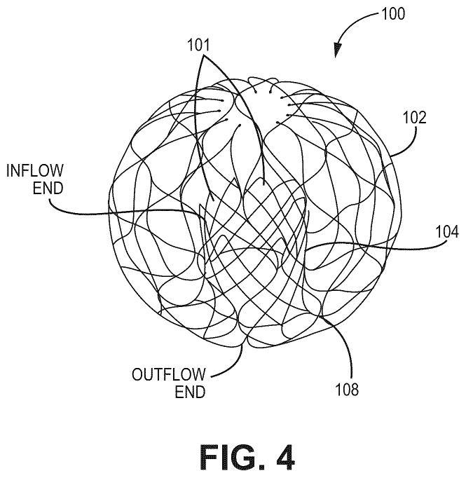

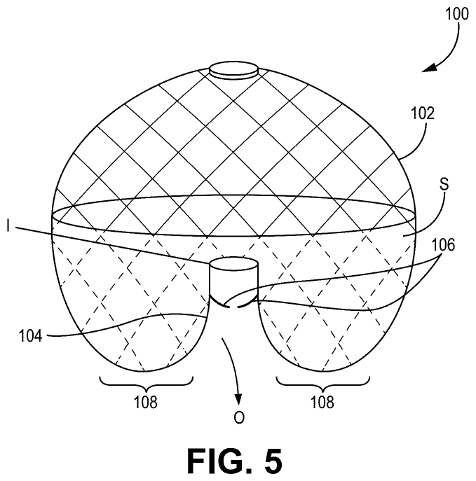

Turning now to FIGS. 4 and 5, an exemplary embodiment is illustrated of an expandable and collapsible prosthetic heart valve stent 100 may comprise an outer section 102 that may generally look like a ball when undeformed and fully expanded and an inner valve support section 104, adapted to support and retain prosthetic valve leaflets 106 within the inner valve support section 104, most preferably at a point that located above the native annulus, and spaced away or above the native leaflets, as shown, though other attachment points for the prosthetic leaflets 106 are within the scope of the present invention. Inner valve support 104 may be cylindrical, but in a preferred embodiment may also be at least partially conical with a larger diameter at an outflow end O than the diameter across at least portions of an inflow end I, wherein the inflow end I is disposed radially inside the outer frame section and wherein the outflow end may define a lower end or edge of the valve support 104. Thus, in a purely conical arrangement, the valve support section 104 may comprise a smoothly decreasing diameter thereacross and this smooth diameter decrease may extend from the outflow end O to the inflow end I. In other embodiments, the inflow end I may comprise one or more lobes extending radially outwardly and that interrupt the smooth conical profile. A preferred embodiment in this regard provides one lobe for each prosthetic leaflet 106 attached within the inner valve support 104 to allow for fuller freedom of movement and improved coaptation.

A preferred construction comprises the prosthetic leaflets 106 disposed or spaced above the native leaflets when the prosthetic valve stent device 100 is implanted, wherein the prosthetic leaflets 106 are attached and spaced sufficiently away from (above) the native leaflets so as to not physically interfere or interact with the native leaflets and the resulting blood flow.

The layer of stent cells that transition from the outer section to the inner section of the stent are termed as transition cells forming a transition section 108 generally as illustrated in FIGS. 4 and 5.

The outer and inner sections of the stent may be constructed from one continuous structure or may combine two or more structures to achieve intended design goals. As known in the art, stent structures may be formed using complementary shaped mandrels, including the outer section 102 of the stent, the transition section 108, and the inner valve support 104—including lobes L discussed above in certain embodiments—as a single unitary structure.

In certain embodiments, the outer support structure may be positioned generally so that it engages with tissue and works to prevent paravalvular leakage (PVL). For example, the outer support structure of the prolapse prevention structure may engage, or be integrated with, the transition section described above to provide a barrier against PVL.

Further in this regard, a preferred embodiment of the device shown in the Figures comprises a skirt S, comprising fabric or tissue, disposed along a portion of the outer surface of the outer frame element 102 and that extends along the outer surface of the transition section 108 and along the inner surface, or inwardly facing surface, of the inner valve support 102 so that the skirt S is facing the flow channel defined therein from the inflow end I to the outflow end O.

The embodiment of FIG. 4, and certain embodiments of FIG. 5, comprises a top-most (or upstream) set of stent cells 101 in the valve support 104 that are generally directed in the upstream direction. Stated differently, the top-most set of stent cells of valve support 104 define the top of the valve support 104.

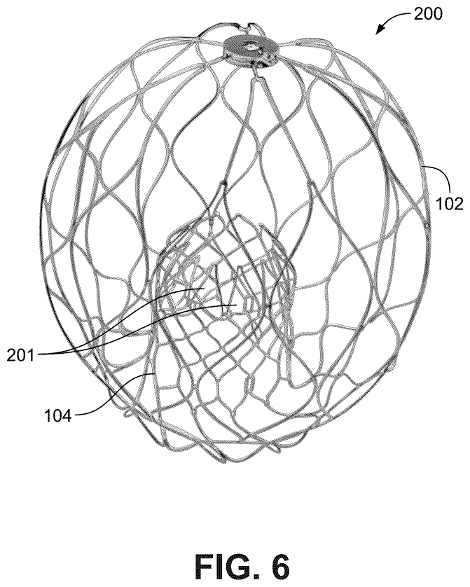

Turning now to FIG. 6, a modified and improved prosthetic heart valve device 200 is provided with the same elements as described above in connection with FIGS. 4 and 5, except that the top-most (upstream) stent cells 101 of the valve support 104 now bent radially inward at 201 and over the inflow end of the valve support 104 and the defined blood flow channel therethrough.

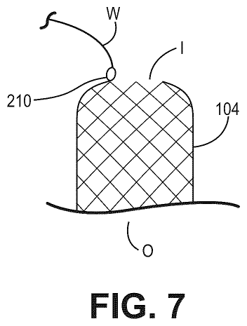

FIG. 7 illustrates a recapture assist mechanism, e.g., a paddle or slot at 210 disposed on, or defined by, one or more of inwardly bent stent cells 201. A single recapture assist mechanism 210 is shown, but more than one may be provided as well. The recapture assist mechanism(s) 210 may be used to receive a wire, e.g., a push and/or pull wire W as is well known in the art, that may be looped or otherwise engage the recapture assist mechanism(s) 210 for aid in directional positioning during delivery and expansion, during recapture/resheathing, or in directional positioning and expanded implantation following recapture/resheathing.

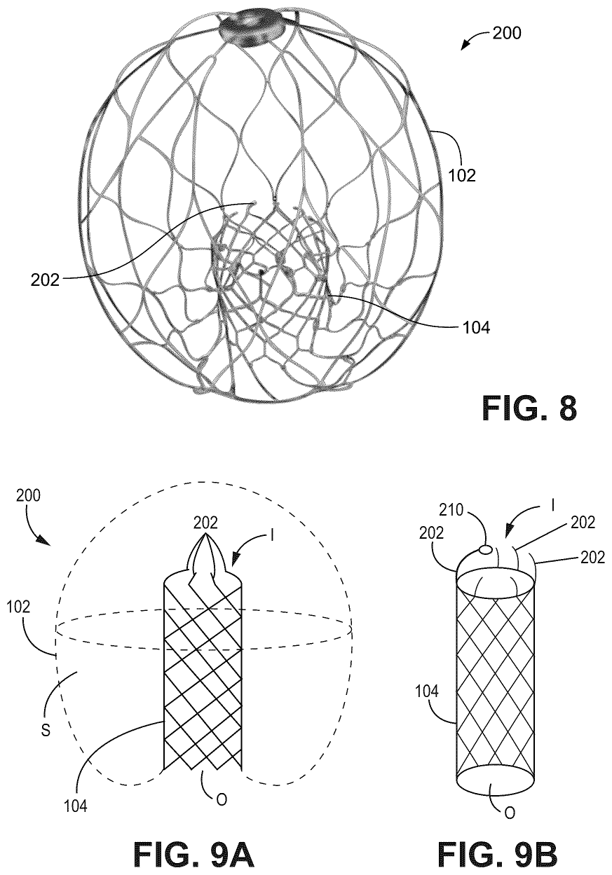

Alternatively, as shown in FIGS. 8 and 9A-9B, a modified and improved heart valve device 200 is provided with the same elements as described above in connection with FIGS. 4-6, except that that the top-most (upstream) stent cells 101 of the valve support 104 now comprise single struts 202 that are bent radially inward and over the inflow end of the valve support 104 and the defined blood flow channel therethrough.

FIG. 9B further illustrates recapture assist mechanism, e.g., a paddle or slot 210 as described above, disposed on the end (upstream and free) portion of one or more of single struts 202. A single recapture assist mechanism 210 is shown, but more than one may be provided. The recapture assist mechanism(s) 210 may be used to receive a wire that may be looped around or through or otherwise engage recapture assist mechanism(s) 210 for aid in directional positioning during delivery and expansion, during recapture and/or resheathing, or in directional positioning and expanded implantation following recapture and/or resheathing.

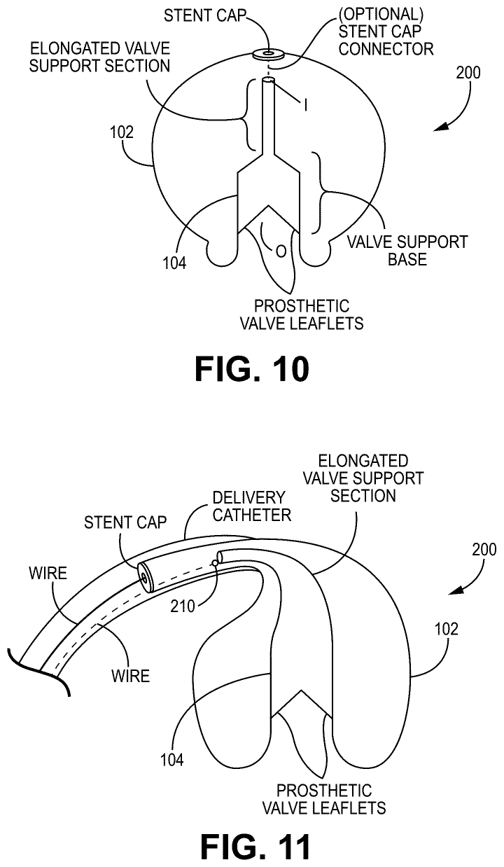

Turning now to FIG. 10, a modified and improved prosthetic heart valve device 200 is provided with the same elements as described above in connection with FIGS. 4 and 5, except that the valve support 104 now comprises an elongated valve support section disposed upstream of the valve support base.

The elongated valve support section may be connected to the stent cap by a stent cap connector such as a wire or strut or other equivalent connecting mechanism or structure. The stent cap comprises a hole or slot for engaging a delivery wire such as a push and/or pull wire to aid in delivery and recapture and/or repositioning of the collapsible and expandable stent device 200.

In addition to an optional connection between the elongated valve support and the stent cap as shown in FIG. 10, FIG. 11 illustrates optional recapture assist and/or delivery assist mechanism at 210, as described herein, which may be a loop or an open paddle and that is adapted for receiving and/or engaging a wire that extends through lumen of delivery catheter to aid in manipulating the elongated valve support section. Mechanism 210 may be attached or formed at any point along the length (or at the top-most or upstream end) of elongated valve support section.

Skirt S (not shown in FIG. 10 or 11) may cover the inner portion of valve support 104 (thereby forming at least part of the blood flow channel defined therein). In some embodiments, the elongated section of the valve support 104 may comprise skirt S on the inner section or may not comprise skirt S.

In some embodiments, the elongated valve support section is in fluid communication with the valve support base and may admit blood into valve support. In this case, the defined blood flow channel may comprise at least part of the elongated valve support section in combination with the valve support base.

FIG. 11 illustrates delivery of the partially expanded prosthetic heart valve device 200 out of a delivery catheter lumen and into a subject heart chamber, e.g., the left atrium. As shown, a transseptal delivery technique may comprise the outer section 102 of the device 200 being expanded and fully deployed while at least a portion of the elongated valve support section, and the upper portion of the outer section 102 including the stent cap, may remain within the lumen of the delivery catheter. The embodiment shown does not provide a connection between the stent cap and the elongated valve support section, but does provide a wired connection with the stent cap and the proximally located operator, as well as recapture and/or delivery assist mechanism 210 connected or engaged wire also extending through lumen of the delivery catheter to a proximally located operator.

Thus, if recapture or resheathing is required, the entire device 200 may be efficiently recaptured since the elongated valve support section are not engaging with the delivery catheter.

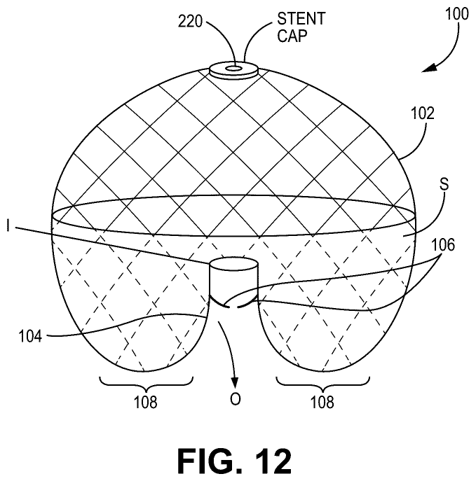

Turning now to FIG. 12, an exemplary embodiment is illustrated of an expandable and collapsible prosthetic heart valve stent 100 may comprise an outer section 102 that may generally look like a ball when undeformed and fully expanded and an inner valve support section 104, adapted to support and retain prosthetic valve leaflets 106 within the inner valve support section 104, most preferably at a point that located above the native annulus, and spaced away or above the native leaflets, as shown, though other attachment points for the prosthetic leaflets 106 are within the scope of the present invention. Inner valve support 104 may be cylindrical, but in a preferred embodiment may also be at least partially conical with a larger diameter at an outflow end O than the diameter across at least portions of an inflow end I, wherein the inflow end I is disposed radially inside the outer frame section and wherein the outflow end may define a lower end or edge of the valve support 104. Thus, in a purely conical arrangement, the valve support section 104 may comprise a smoothly decreasing diameter thereacross and this smooth diameter decrease may extend from the outflow end O to the inflow end I. In other embodiments, the inflow end I may comprise one or more lobes extending radially outwardly and that interrupt the smooth conical profile. A preferred embodiment in this regard provides one lobe for each prosthetic leaflet 106 attached within the inner valve support 104 to allow for fuller freedom of movement and improved coaptation.

A preferred construction comprises the prosthetic leaflets 106 disposed or spaced above the native leaflets when the prosthetic valve stent device 100 is implanted, wherein the prosthetic leaflets 106 are attached and spaced sufficiently away from (above) the native leaflets so as to not physically interfere or interact with the native leaflets and the resulting blood flow.

The layer of stent cells that transition from the outer section to the inner section of the stent are termed as transition cells forming a transition section 108 generally as illustrated in FIG. 12.

The outer and inner sections of the stent may be constructed from one continuous structure or may combine two or more structures to achieve intended design goals. As known in the art, stent structures may be formed using complementary shaped mandrels, including the outer section 102 of the stent, the transition section 108, and the inner valve support 104—including lobes L discussed above in certain embodiments—as a single unitary structure.

In certain embodiments, the outer support structure may be positioned generally so that it engages with tissue and works to prevent paravalvular leakage (PVL). For example, the outer support structure of the prolapse prevention structure may engage, or be integrated with, the transition section described above to provide a barrier against PVL.

Further in this regard, a preferred embodiment of the device shown in the Figures comprises a skirt S, comprising fabric or tissue, disposed along a portion of the outer surface of the outer frame element 102 and that extends along the outer surface of the transition section 108 and along the inner surface, or inwardly facing surface, of the inner valve support 102 so that the skirt S is facing the flow channel defined therein from the inflow end I to the outflow end O.

The embodiment of FIG. 4, and certain embodiments of FIG. 5, comprises a top-most (or upstream) set of stent cells 101 in the valve support 104 that are generally directed in the upstream direction. Stated differently, the top-most set of stent cells of valve support 104 define the top of the valve support 104.

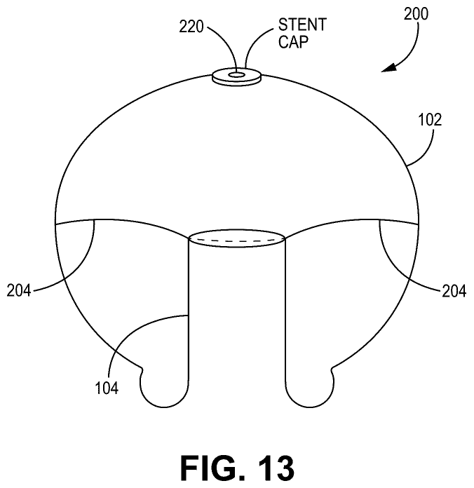

Turning now to FIG. 13, a modified and improved prosthetic heart valve device 200 is provided with the same elements as described above in connection with FIG. 12, except that the valve support 104 now comprises at least one, and preferably two (though more may be used) connectors 204 extending between the valve support 104 and the outer section 102 of stent device 200. As shown, the connectors 201 are connected at a first end to the valve support 104 at its top-most or upstream end, and at a second end connected to the outer section 102 of device 200. The connector(s) 204 may be heat set or may use a shape memory or other material as the skilled artisan will appreciate. The connector(s) 204 may be generally straight when connected and the device 200 is fully expanded, or they may be slightly bent to provide additional outward expanding force on the stent 102 which can assist in ensuring the implanted stent device 200 remains in place against the chamber walls. As shown, the connector(s) 204 are disposed within the interior defined by the outer section 102.

The first and second ends of the connector(s) 204 may be mechanically or otherwise attached to, or formed from, the valve support 104 and/or the outer section 102 of device 200. Exemplary connection methods, all well known to the artisan, include but are certainly not limited to, suturing, welding, riveting.

The stent cap shown in FIGS. 12 and 13 comprises a hole or slot 220 for engaging a delivery wire such as a push and/or pull wire to aid in delivery and recapture and/or repositioning of the collapsible and expandable stent device 200.

The various structural modifications described herein allow the valve support 104 to collapse inwardly more efficiently during both loading of the device 200 into the lumen of a delivery catheter as well as at least partial recapture when necessary of an at least partially expanded device 200 in order to reposition and/or reexpand and/or reimplant the device 200.

Though this structure may be most advantageous in a transseptal delivery technique and access to, e.g., the left atrium and mitral valve, other delivery access routes, techniques and heart valves may also be amenable to the improved structure and related advantages of device 200.

It is noteworthy that the various embodiments of the presently described prosthetic valve stent device 200 may be delivered percutaneously via one of at least the following known access and delivery routes: femoral access, venous access, trans-apical, trans-aortic, trans-septal, and trans-atrial, retrograde from the aorta delivery techniques. Alternatively, the prosthetic valve stent device 200 may be delivered and implanted using surgical and/or open heart techniques.

Various embodiments of the inventive concepts described herein comprise:

1. A prosthetic heart valve device, adapted for expansion and implantation into a heart chamber of a patient for supplementation and/or replacement of native valve leaflet functionality, the device comprising:
a collapsible and expandable stent having an outer section comprising an outer surface, an inner surface, and defining an interior;
a valve support operationally connected to the collapsible and expandable stent, wherein the valve support extends radially upward into the interior of the outer section and comprising an inflow end and an outflow end, the inflow end extending radially upward into the outer section, wherein a blood flow channel is defined between the inflow and outflow ends, and wherein the valve support is inverted entirely within the interior of the outer section,
a plurality of prosthetic valve leaflets disposed within the blood flow channel defined by the valve support section, wherein prosthetic valve leaflets are configured to allow flow from the inflow end to the outflow end of the flow channel and prevent flow from the outflow end of the flow channel to the inflow end of the flow channel,
wherein the valve support comprises a top section that is defined by a plurality of single struts that are bent radially inwardly over the blood flow channel.

2. The device of embodiment 1, wherein the valve support is defined and formed by the collapsible and expandable stent.

3. The device of embodiment(s) 1 and 2, further comprising transition cells that turn the outer section of the collapsible and expandable stent radially inwardly, the transition cells disposed between the outer section of the collapsible and expandable stent and the valve support.

4. The device of embodiment(s) 1-3, wherein the valve support and outer section of the valve support comprise a single unitary stent.

5. The device of embodiment(s) 1-4, wherein at least one of the plurality of single struts comprises a recapture assist mechanism.

6. The device of embodiment 5, wherein the recapture assist mechanism comprises an open paddle adapted for receiving a wire therethrough.

7. A method for delivery, recapturing and/or positioning the collapsible and expandable prosthetic heart valve device of embodiment(s) 1-6 into a heart chamber of a patient, comprising:
providing a delivery catheter having a lumen therethrough;
placing the delivery catheter within the patient such that a distal end of the delivery catheter and lumen are within the heart chamber and a proximal end of the delivery catheter and lumen are outside the patient's body;
collapsing and loading the prosthetic heart valve device into a proximal end of the delivery catheter lumen and translating the prosthetic heart valve device distally;
delivering the prosthetic heart valve device from the distal end of the delivery catheter lumen at least partially into the heart chamber;
at least partially expanding the prosthetic heart valve device within the heart chamber; and
when properly positioned, implanting the prosthetic heart valve device within the chamber.

8. The method of embodiment 7, further comprising providing at least one push and/or pull wire comprising a distal end that is operatively engaged with at least one of the recapture assist mechanism(s).

9. The method of embodiment 8, further comprising manipulating the push and/or pull wire(s) to modify the position of at least the valve support during expansion, positioning, recapturing and/or implanting of the prosthetic heart valve device.

10. The method of embodiment(s) 7-9, wherein the heart chamber comprises the left atrium.

11. The method of embodiment 10, wherein the prosthetic heart valve device is implanted and adapted to supplement the native mitral valve leaflet functionality.

12. The method of embodiment(s) 10 and 11, wherein the prosthetic heart valve device is implanted and adapted to supplement and eventually fully replace the native mitral valve leaflet functionality.

13. The method of embodiment(s) 7-10 wherein the delivery catheter is placed within the patient using the transseptal delivery access route.

14. The method of embodiment(s) 7-10, wherein the delivery catheter is placed within the patient using one of the access routes in the group consisting of: transapical; transfemoral; transatrial.

Additional embodiments of the various inventive concepts described herein further comprise:

1. A prosthetic heart valve device, adapted for expansion and implantation into a heart chamber of a patient for supplementation and/or replacement of native valve leaflet functionality, the device comprising:
a collapsible and expandable stent having an outer section comprising an outer surface, an inner surface, and defining an interior and comprising a stent cap disposed at an upper portion of the outer section, the stent cap adapted to connect with a delivery wire;
a valve support operationally connected to the collapsible and expandable stent, wherein the valve support extends radially upward into the interior of the outer section and comprising an inflow end and an outflow end, the inflow end extending radially upward into the outer section, wherein a blood flow channel is defined between the inflow and outflow ends, and wherein the valve support is inverted entirely within the interior of the outer section,
a plurality of prosthetic valve leaflets disposed within the blood flow channel defined by the valve support section, wherein prosthetic valve leaflets are configured to allow flow from the inflow end to the outflow end of the flow channel and prevent flow from the outflow end of the flow channel to the inflow end of the flow channel,
wherein the valve support comprises an elongated section extending in the upstream direction within the interior of the outer section.

2. The device of embodiment 1, wherein the valve support is defined and formed by the collapsible and expandable stent.

3. The embodiment(s) of claims 1 and 2, further comprising transition cells that turn the outer section of the collapsible and expandable stent radially inwardly, the transition cells disposed between the outer section of the collapsible and expandable stent and the valve support.

4. The device of embodiments 1-3, wherein the valve support and outer section of the valve support comprise a single unitary stent.

5. The device of embodiment(s) 1-4, wherein the elongated section of the valve support is operatively connected with the stent cap.

6. The device of embodiment(s) 1-5, wherein the elongated section of the valve support comprises a recapture and/or delivery assist mechanism disposed or defined thereon.

7. The device of embodiment 6, wherein the recapture assist mechanism comprises an open loop or open paddle adapted for receiving a wire therethrough.

8. A method for delivery, recapturing and/or positioning the collapsible and expandable prosthetic heart valve device of claims 1-7 into a heart chamber of a patient, comprising:
providing a delivery catheter having a lumen therethrough;
placing the delivery catheter within the patient such that a distal end of the delivery catheter and lumen are within the heart chamber and a proximal end of the delivery catheter and lumen are outside the patient's body;
collapsing and loading the prosthetic heart valve device into a proximal end of the delivery catheter lumen and translating the prosthetic heart valve device distally;
delivering the prosthetic heart valve device from the distal end of the delivery catheter lumen at least partially into the heart chamber;
at least partially expanding the prosthetic heart valve device within the heart chamber, wherein a portion of the elongated valve support section remains within the delivery catheter lumen; and
when properly positioned, implanting the prosthetic heart valve device within the chamber.

9. The method of embodiment 8, further comprising providing at least one push and/or pull wire comprising a distal end that is operatively engaged with at least one of the recapture assist mechanism(s); and adjusting or manipulating the position of the elongated valve support section with the at least one push and/or pull wire.

10. The exemplary method of embodiment 9, further comprising manipulating the push and/or pull wire(s) to modify the position of at least the valve support during expansion, positioning, recapturing and/or implanting of the prosthetic heart valve device.

11. The method of embodiment(s) 7-10, wherein the heart chamber comprises the left atrium.

12. The method of embodiment 11 wherein the prosthetic heart valve device is implanted and adapted to supplement the native mitral valve leaflet functionality.

13. The method of embodiment(s) 11 or 12, wherein the prosthetic heart valve device is implanted and adapted to supplement and eventually fully replace the native mitral valve leaflet functionality.

14. The method of embodiment(s) 7-13 wherein the delivery catheter is placed within the patient using the transseptal delivery access route.

15. The method of embodiment(s) 7-13, wherein the delivery catheter is placed within the patient using one of the access routes in the group consisting of: transapical; transfemoral; and transatrial.

Still further additional embodiments of the various inventive concepts described herein further comprise:

1. A prosthetic heart valve device, adapted for expansion and implantation into a heart chamber of a patient for supplementation and/or replacement of native valve leaflet functionality, the device comprising:
a collapsible and expandable stent having an outer section comprising an outer surface, an inner surface, and defining an interior and comprising a stent cap disposed at an upper portion of the outer section, the stent cap adapted to connect with a delivery wire;
a valve support operationally connected to the collapsible and expandable stent, wherein the valve support extends radially upward into the interior of the outer section and comprising an inflow end and an outflow end, the inflow end extending radially upward into the outer section, wherein a blood flow channel is defined between the inflow and outflow ends, and wherein the valve support is inverted entirely within the interior of the outer section,
a plurality of prosthetic valve leaflets disposed within the blood flow channel defined by the valve support section, wherein prosthetic valve leaflets are configured to allow flow from the inflow end to the outflow end of the flow channel and prevent flow from the outflow end of the flow channel to the inflow end of the flow channel; and
at least one collapsible and expandable connecting strut disposed between the valve support and the outer section, the at least one connecting strut located in the interior of the outer section and connected with the valve support at a first end and with the outer section at a second end.

2. The device of embodiment 1, wherein the valve support is defined and formed by the collapsible and expandable stent.

3. The device of embodiment(s) 1 and 2, further comprising transition cells that turn the outer section of the collapsible and expandable stent radially inwardly, the transition cells disposed between the outer section of the collapsible and expandable stent and the valve support.

4. The device of embodiment(s) 1-3, wherein the valve support and outer section of the collapsible and expandable stent comprise a single unitary stent.

5. The device of embodiment(s) 1-4, wherein the at least one collapsible and expandable connecting strut is heat set in position.

6. The device of embodiment(s) 1-5 wherein the at least one collapsible and expandable strut is formed from the valve support or the outer section of the collapsible and expandable stent.

7. The device of embodiment(s) 1-6, wherein at least one end of the at least one collapsible and expandable strut is connected to the valve support and/or the outer section of the collapsible and expandable stent.

8. The device of embodiment(s) 1-7, wherein the at least one connecting strut is operatively disposed at, or proximate, the inflow end of the valve support.

9. The device of embodiment(s) 1-7, wherein the at least one connecting strut is operatively disposed along the valve support at a location between the inflow end and the outflow end of the valve support.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1. A prosthetic heart valve device, adapted for expansion and implantation into a heart chamber of a patient, the device comprising:
a collapsible and expandable stent having:
an outer section defining an interior; and
a valve support that:
extends upward into the interior; and
comprises an inflow end and an outflow end, wherein a blood flow channel is defined between the inflow and outflow ends, and wherein the valve support is inverted entirely within the interior; and
a plurality of prosthetic valve leaflets disposed within the blood flow channel and configured to allow flow from the inflow end to the outflow end and prevent flow from the outflow end to the inflow end;
wherein:
the inflow end of the valve support comprises a plurality of top-most stent cells that are bent inwardly over the blood flow channel.

2. The device of claim 1 wherein the valve support is defined by the collapsible and expandable stent.

3. The device of claim 1 further comprising transition cells that turn the outer section of the collapsible and expandable stent inwardly, the transition cells being disposed between the outer section of the collapsible and expandable stent and the valve support.

4. The device of claim 2 further comprising transition cells that turn the outer section of the collapsible and expandable stent inwardly, the transition cells being disposed between the outer section of the collapsible and expandable stent and the valve support.

5. The device of claim 1 wherein the valve support and outer section of the valve support comprise a single unitary stent.

6. The device of claim 1 wherein at least one of the plurality of top-most stent cells comprises a recapture assist mechanism.

7. The device of claim 6 wherein the recapture assist mechanism is configured to engage a wire to assist in positioning the device in the heart chamber.

8. The device of claim 7 wherein the wire comprises a push and/or pull wire.

9. The device of claim 8 wherein the recapture assist mechanism comprises an open paddle configured to engage the wire.

10. The device of claim 8 wherein the recapture assist mechanism comprises a slot configured to engage the wire.

11. A method for delivery, recapture and/or positioning of the prosthetic heart valve device of claim 1 into a heart chamber of a patient, the method comprising:
providing a delivery catheter having a lumen therethrough;
placing the delivery catheter within the patient such that a distal end of the delivery catheter is within the heart chamber and a proximal end of the delivery catheter is outside the patient;
collapsing and loading the prosthetic heart valve device into a proximal end of the delivery catheter lumen and translating the prosthetic heart valve device distally;
delivering the prosthetic heart valve device from the distal end of the delivery catheter at least partially into the heart chamber;
at least partially expanding the prosthetic heart valve device within the heart chamber; and
when properly positioned, implanting the prosthetic heart valve device within the heart chamber.

12. The method of claim 11 further comprising providing at least one wire comprising a distal end that is operatively engaged with a recapture assist mechanism.

13. The method of claim 12 further comprising manipulating at least one of the wires to modify a position of at least the valve support in the heart chamber.

14. The method of claim 11 wherein the heart chamber is a left atrium.

15. The method of claim 11 wherein, when the prosthetic heart valve device is implanted in the heart chamber and the heart chamber is a left atrium, the prosthetic heart valve device is configured to supplement native mitral valve leaflet functionality.

16. The method of claim 11 wherein, when the prosthetic heart valve device is implanted in the heart chamber and the heart chamber is a left atrium, the prosthetic heart valve device is configured to replace native mitral valve leaflet functionality.

17. The method of claim 11 wherein the delivery catheter is configured to be placed within the patient using a transseptal delivery access route.

18. The method of claim 11 wherein the delivery catheter is configured to be placed within the patient using a transapical access route.

19. The method of claim 11 wherein the delivery catheter is configured to be placed within the patient using a transfemoral access route.

20. The method of claim 11 wherein the delivery catheter is configured to be placed within the patient using a transatrial access route.
